(19) United States
(12) Patent Application Publication
(10) Pub. No.: US 2017/0087313 A1
(43) Pub. Date: Mar. 30, 2017

(54) SPACER FOR INHALATION OF MEDICINAL AEROSOL PRODUCTS AND PHARMACEUTICAL SOLUTIONS FOR THE TREATMENT OF RESPIRATORY DISEASES

(71) Applicants: AEROMEDICAL S. A., Ciudad de Buenos Aires (AR); Laboratorio Pablo Cassará S.R.L., Buenos Aires (AR)

(72) Inventors: Julián Esteban ROSENFELD, Ciudad de Buenos Aires (AR); Jorge Alberto CASSARA, Buenos Aires (AR)

(73) Assignees: AEROMEDICAL S. A., Ciudad de Buenos Aires (AR); Laboratorio Pablo Cassará S.R.L., Buenos Aires (AR)

(21) Appl. No.: 15/276,889

(22) Filed: Sep. 27, 2016

(30) Foreign Application Priority Data
Sep. 30, 2015 (AR) M20150103159

(51) Int. Cl. A61M 15/00; A61M 16/06

(52) U.S. Cl. CPC A61M 15/0086; A61M 15/009; A61M 15/0013; A61M 16/06; A61M 2230/40; A61M 2205/583

(57) ABSTRACT

The present invention relates to an aerochamber for inhalation of medicinal products in aerosol for the treatment of respiratory ailments. The aerochamber includes a tubular body including a proximal end with a front cover adapted to mount a mask to cover a mouth and a nose of a patient; a distal end with a rear cover adapted to connect to a container with a pressurized pharmaceutical solution to be inhaled, the rear cover includes an annular wall including a hole that matches with an opening on the distal end; a translucent cap having a front wall, an inside wall, a cavity, an opening on the front wall, the translucent cap is placed on a section of the annular wall of the rear cover; an inhalation sensor housed on the cavity of the translucent cap. The inhalation sensor includes an indication valve having: a flexible tab, the flexible tab has a lower end secured to the rear cover, the flexible tab is located between the opening of the front wall of the translucent cap and the hole of the annular wall of the rear cover; a flange having an inverted U shape secured to a section of the flexible tap; and an upper flange projecting from the inside wall of the translucent cap and defining a bumper limiting a maximum angular displacement of the flexible tab in a working position.

### FIELD OF THE INVENTION

The present invention refers to an aerochamber for inhalation of medicinal aerosol products and pharmaceutical solutions for the treatment of respiratory diseases, and more particularly to an aerochamber with an inhalation sensor that makes it easy to detect the proper aspiration of the medicinal product, preferably designed for infants, children, people with motor skills problems or with diminution of their control power in the mouth muscles for the aspiration, and in general to people who have not established or do not respond to a proper breathing pattern, or have difficulty in coordination between the shooting of medicinal spray and the inhalation, and, therefore, it is necessary to ensure that they properly aspire the pharmacological substance or medicine to be supplied to treat their respiratory condition.

### BACKGROUND OF THE INVENTION

For the administration of the medicinal and pharmaceutical products in spray to people with respiratory conditions is frequent in the use of aerochambers, in which at the proximal end is generally placed a one-way inhalation valve and is attached to a removable mask that rests on the face of the patient, covering his mouth and nose, and at the distal end is placed a small container including the product to be inhaled. The function of the unidirectional inhalation valve normally closed, while maintaining the ‘cloud’ of the medicine inside the tubular body of the aerochamber, and opens with each inhalation of the patient allowing the passage of the medication to the airways. The inhalation valve helps the medication to better reach the lower airways and for the medication to be deposited less in the patient's throat, which avoids the side effects of some drugs at this level. Therefore, the correct operation of the inhalation valve is crucial to achieve the efficient administration of the drugs through the use of these types of devices.

However, the experience in the use of these aerochambers has put in evidence that in certain patients, especially babies and children, but also adults that do not respond to a proper pattern in the intake and exhalation functions, often happens that the inhalations are not performed with sufficient intensity and regularity to get the prescribed medicine to reach the lower airways and, as a result, the treatment of the respiratory conditions fails.

In order to solve the previously mentioned disadvantages, in order that an assistant of the patient will be able to verify if the inhalation is properly done, it has been developed aerochambers that include a sensor that allows control and visually checks the step of inhalation. For example, such aerochambers can be mentioned by disclosing the U.S. Pat. No. 8,550,067 B2 to Trudell Medical International and others. This aerochamber incorporates in its proximal end, i.e. in the adjacencies of the adapter of the mask to cover the mouth and nose of the patient, a display cap having inside an aspiration flow indicator, where the indicator is essentially a mobile tab, or tap sensor of the aspiration, able to oscillate between a resting position and an angular position moved in response to the action of inhalation by the patient. The angle of displacement of the sensor tab varies depending on the inhalation strength and, therefore, if that strength is weak or is interrupted prematurely, the angular movement of the tab will not reach its maximum displacement, which will indicate and detect that the patient did not inhale enough to receive the necessary dose of the product that must reach the airways. While the inhalation sensor of this aerochamber constitutes a remarkable technical and practical contribution to control if the inhalation is performed correctly, for being the sensor placed near the mask of the aerochamber, the display of the indicator tab is usually difficult, precisely, by its location being very close to the face the patient and, if the patient or other person holds the aerochamber from the tubular body of the same or from the mask with one hand, can block or prevent the display of the status and movement of the tab by those who assist the patient.

It is also worth mentioning that a major drawback of the aerochamber of the U.S. Pat. No. 8,550,067 B2 is that the tab only measures the inhalation from the patient, but does not ensure the opening of the inhalation valve of the aerochamber, which represents the critical issue for the correct treatment of the patient. This is so because the tab operates independently of the aerochamber inhalation valve and, consequently, there is no way of ensuring that the movement of the tab indicates correct inhalation of medication by the user. In an extreme case, the inhalation valve of the aerochamber could be damaged, blocked, or even sealed, and the patient would inhale only the outside air that the tab allows to pass, and this would indicate inhalation, which constitutes an important functional deficiency of the aerochamber. This situation is particularly dangerous, since the patient would not be receiving the medication, which would be unnoticed since the movement of the tab would assume that the inhalation is correct.

### SUMMARY OF THE INVENTION

For resolving the inconvenient previously mentioned about existing aerochambers with inhalation sensors, the aerochamber object of the present invention has been developed, which allows to easily display the behavior of the sensor even when the patient or another person manually holds the aerochamber from its tubular body or from the mask. In addition, the aerochamber of the present invention allows to safely verify the occurrence of the inhalation of the content of the aerochamber, promoting an effective treatment. The tab of this aerochamber will be displaced only if the inhalation valve of the aerochamber is first defeated by the inhalation of the patient. That is, if the patient fails to open the inhalation valve, whether from suffering from a severe obstructive pathology, or by being the aerochamber inhalation valve damaged, the tab will not move, thus indicating that the product did not extract from inside the aerochamber, which can be immediately displayed. In other words, in the aerochamber of the present invention the movement of the tab is functionally related to the inhalation valve, and the tab can move by pulling the vacuum generated by inhalation only when the inhalation valve opens first. In this way, the movement of the tab sensor ensures that the patient has inhaled the contents of the aerochamber.

Another advantage is that what is aspirated by the patient comes entirely from the content in the tubular body of the aerochamber, i.e. air with medication, since although the sensor tab takes air from the outside, the air then passes to the tubular body joining the medication flow extracted from the corresponding container. This gives a high degree of efficiency and reliability of the aerochamber for the treatment to be carried out.

It is therefore the object of the present invention to provide an inhalation aerochamber for aerosol pharmaceutical solutions and medicinal products for the treatment of respiratory diseases, of the type comprising a tubular body whose proximal end has a front cover that contains an inhalation valve where attaches a mask that covers the mouth and nose of the patient for the inhalation, and at the distal end of the tubular body includes a rear cover which fits the container of a pressurized substance to be inhaled, including the aerochamber an inhalation sensor designed to visually detect the correct inhalation the pharmaceutical solutions and/or medicinal to inhale. The inhalation sensor is arranged in the distal end of the tubular body, and more precisely on a section of the rear cover. The sensor includes a translucent cap housing an inhalation detection valve defined by a flexible tab inhalation indicator extending perpendicularly from this section on the rear cover, a “U” inverted shaped flange arranged ahead of the flexible tab defining an airtight support of a perimeter section of the tab, and a flange disposed behind the tab by defining a limited stop of the maximum displacement of the same. The front wall of the cap, the section on the rear cover, and the distal end of the tubular body present respective openings for the passage of a flow of outside air which moves the tab angularly when the inhalation of the pressurized product in the container connected to the nozzle of the rear cover is performed correctly.

### BRIEF DESCRIPTION OF THE DRAWINGS

For greater clarity and understanding of the object of the present invention, it has been illustrated in Figures representing the preferred form of preparation, where:

FIG. 1 shows a side view of the aerochamber;

FIG. 2 shows a partial enlarged view and in frontal perspective of the distal end of the aerochamber;

FIG. 3 shows a partial expanded view and on rear perspective of the distal end of the aerochamber;

FIG. 4 shows a perspective front view on the rear cover of the tubular body of the aerochamber including the inhalation sensor;

FIG. 5 shows a perspective rear view showing the rear cover of the aerochamber and the inhalation sensor cap;

FIG. 6 shows a partial perspective back view of the distal end of the aerochamber showing a cut of the inhalation sensor cap;

FIG. 7A shows a side view of the distal end of the aerochamber showing the sensor in normal resting position;

FIG. 7B shows a side view of the distal end of the aerochamber showing the sensor in an activated position or in a position indicating a correct inhalation; and

FIG. 8 shows a side view of the aerochamber showing the movement of the medicinal product when inhalation sensor is activated and the inhalation valve opens.

### DETAILED DESCRIPTION OF THE INVENTION

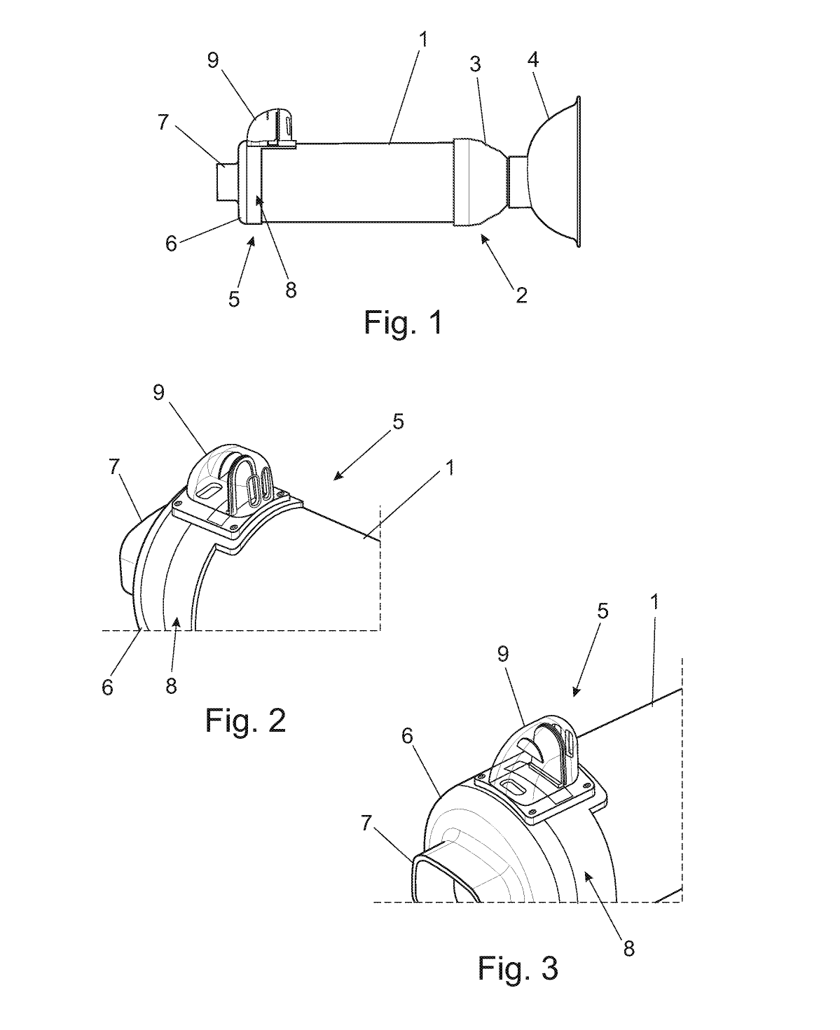

FIGS. 1 to 3 show that the aerochamber for inhalation of pharmaceutical solutions and medicinal products in aerosol or spray includes a body tubular (1), which proximal end 2 includes a front cover 3 adapted for mounting a mask 4 that covers the mouth and the nose of the patient during the inhalation. In the distal end 5 of the body tubular 1 includes a rear cover 6 where is coupled, from the nozzle 7, the container including the pressurized substance to be inhaled.

In the distal end 5 of the aerochamber, and more precisely on a section of the annular wall 8 of the rear cover 6, an inhalation sensor is located which includes a translucent cap 9 and an inhalation indication valve housed in a cavity defined between this section of the annular wall 8 and the inside face of the cap 9.

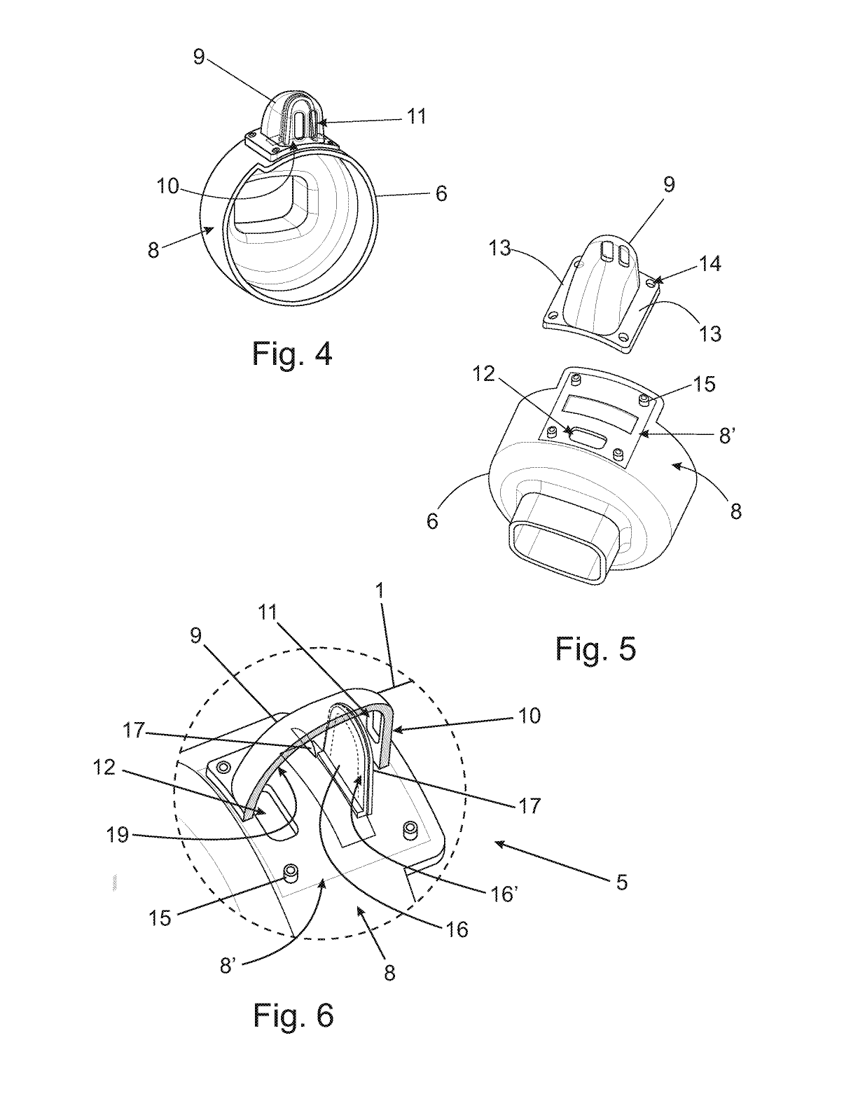

As can clearly be appreciated in FIGS. 4 to 6, the front wall 10 of the cap 9 presents openings 11, while the section 8′ of the annular wall 8 of the rear cover 6, and the distal end 5 of the tubular body 1 presented respective matching openings 12, whose purpose will be explained later.

The cutting of it in FIG. 4 shows from the lower lateral ends of the cap 9 respective fins 13 are extended with holes 14 through which pass corresponding protuberances 15 that project from the section 8′ of the annular wall 8 for anchoring the cap 9. While the cap 9 must be hermetically mounted on the annular wall 8 of the cover 6, still in practice may be removable of the same.

The inhalation indication valve, as can be observed in FIG. 6, is defined by a flexible tab 16, indicator of the inhalation force, whose lower end is fixed to the section 8′ of the rear cover 6 and extends in perpendicular form to the same. The flexible tab 16 is interposed between the openings 11 of the front wall 10 of the cap 9 and the openings 12 of the section 8′ and the distal end 5 of the tubular body 1.

Immediately in front of the flexible tab 16 and parallel to it, the sensor valve includes a fixed flange 17, for example of inverted “U” shape, where jointly support a perimeter section 16′ of the tab 16, while in the back and distanced of the tab, the sensor valve includes an upper flange 18 that projects from the inside wall 19 of the cap 9 and defines a limited stopper of the maximum angular displacement of the tab 16 during the inhalation. The lateral and upper edges of the tab 16 are adjacent to the surface of the inside wall 19 of the cap 9 and complementary with the form of the same.

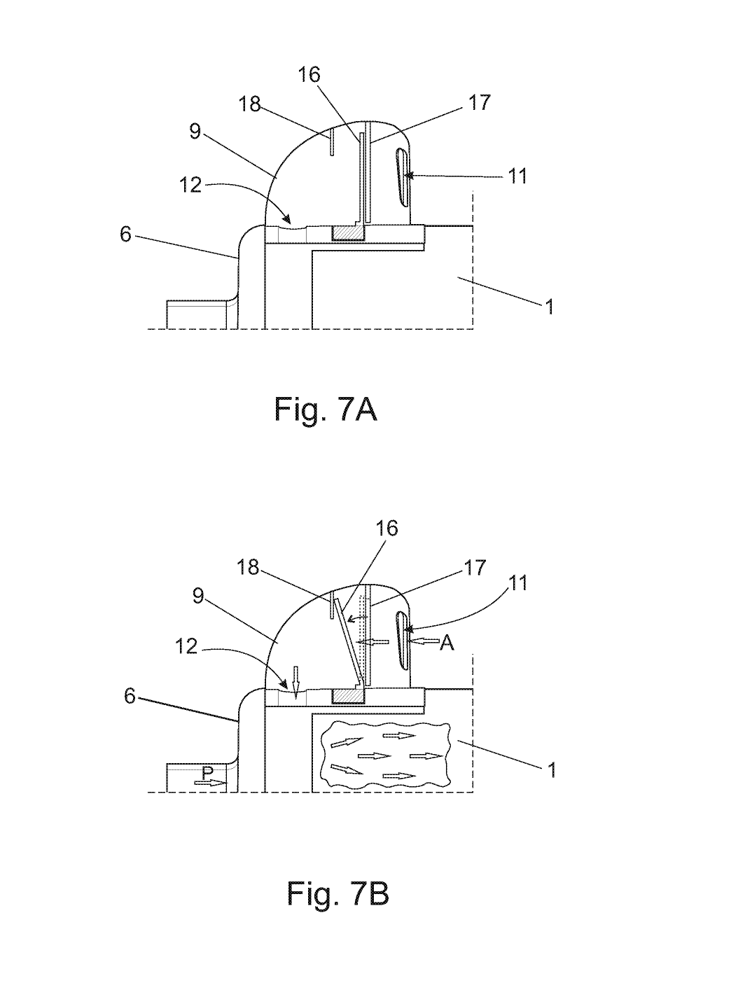

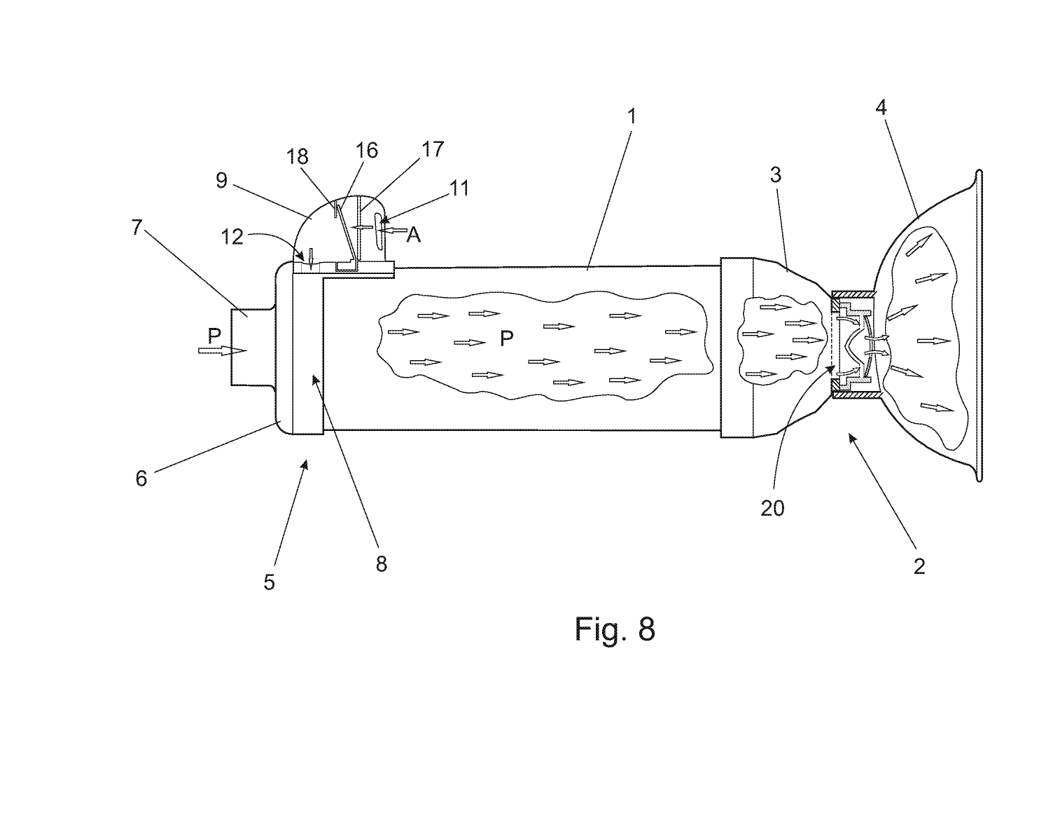

FIGS. 7A and 7B respectively illustrate two positions of sensor tab 16, i.e. closed before the inhalation or when the inhalation force exerted by the patient is very weak, as shown in FIG. 7A, and displaced until its maximum angular position determined by the upper flange 18 when the aspiration force of the patient is enough for the inhalation of the product P being supplied from the aerochamber, as shown in FIG. 7B. The FIG. 8 shows sensor 16 tongue and valve inhalation 20 positions that allow the passage of the medication when the patient correctly performs the aspiration.

In the resting position, the tab 16 stays in tight contact with the flange 17, and when the aspiration force of the patient manages to open the inhalation valve 20 of the aerochamber, overcome this force, and by the effect of the suctioning of the outside air A through openings 11, the tab 16 is displaced until its maximum angular position on the upper flange 18. Of this way, the sensor valve only acts if before the inhalation valve 20 of the aerochamber is overcome, ensuring, by simple observation of the position of the tab 16, if the patient is inhaling the medicament.

Having particularly described and given the nature of the present invention and the form it can be taken to practice, it declares claiming ownership and exclusive rights to:

1-4. (canceled)

5. An aerochamber for inhalation of aerosol or spray pharmaceutical solutions for treatment of respiratory diseases comprising:
a tubular body including a proximal end with a front cover adapted to mount a mask to cover a mouth and a nose of a patient;
a distal end with a rear cover adapted to connect to a container with a pressurized pharmaceutical solution to be inhaled, the rear cover includes an annular wall including a hole that matches with an opening on the distal end;
a translucent cap having a front wall, an inside wall, a cavity, an opening on the front wall, the translucent cap is placed on a section of the annular wall of the rear cover;
an inhalation sensor housed on the cavity of the translucent cap;
wherein the inhalation sensor includes an indication valve having:
a flexible tab, the flexible tab has a lower end secured to the rear cover, the flexible tab is located between the opening of the front wall of the translucent cap and the hole of the annular wall of the rear cover;
a flange having an inverted U shape secured to a section of the flexible tap; and
an upper flange projecting from the inside wall of the translucent cap and defining a bumper limiting a maximum angular displacement of the flexible tab in a working position.

6. The aerochamber according to claim 5, wherein lateral and upper edges of the flexible tab are adjacent to a surface of the inside wall of the cap and have a complementary shape to a shape of the inside wall.

7. The aerochamber according to claim 5, wherein the translucent cap is hermetically mounted on the rear cover.

8. The aerochamber according to claim 5, wherein the translucent cap is removable from the rear cover.

9. The aerochamber according to claim 5, wherein the translucent cap includes lower lateral ends including fins 13 that laterally extended from the translucent cap, the fins include holes through which pass corresponding protuberances that project from the section of the annular wall of the rear cover to anchor the translucent cap to the rear cover.
